(19) United States
(12) Patent Application Publication
(10) Pub. No.: US 2024/0151528 A1
(43) Pub. Date: May 9, 2024

(54) Watershed Autonomous Robotic Data Recorders for Mapping Subsurface Topography and Obtaining Water Quality Data For Bodies of Water

(71) Applicant: CCIWADR, LLC, Columbus, OH (US)

(72) Inventors: Stephen Curless, East Canton, OH (US); Jack Ray, Hilliard, OH (US); Joyce Johnson, Hilliard, OH (US)

(73) Assignee: CCIWADR, LLC, Columbus, OH (US)

(21) Appl. No.: 17/980,551

(22) Filed: Nov. 4, 2022

(51) Int. Cl. G01C 13/00; B63B 1/12; B63B 21/16; B63B 21/66; B63B 79/15; B63H 7/02; G01N 33/18

(52) U.S. Cl. CPC G01C 13/008; B63B 1/121; B63B 21/16; B63B 21/66; B63B 79/15; B63H 7/02; G01N 33/1886; B63B 2211/02

(57) ABSTRACT

A watershed autonomous robotic data recorder maps subsurface topography and obtains water quality data for bodies of water. The device is capable of fully autonomous, manual, or hybrid navigation and includes a modular hull design which enables customization to accommodate both large and small bodies of water. A tangle-proof propulsion system is provided, to propel the hull(s). A deployable sensor pod includes a water sensor for determining if the sensor pod is in water, a pressure sensor for determining depth of the sensor pod, and a plurality of sensors for measuring water conditions. An onboard controller operates a winch that raises and lowers the sensor pod while the controller and a GPS receiver log positions of the sensor pod. This three-dimensional environmental, physical and chemical water quality attribute data collection creates a digital twin of the body of water to establish a common data environment for water modeling and analysis.

### CROSS-REFERENCE TO RELATED APPLICATIONS

Not applicable

### STATEMENT REGARDING FEDERALLY SPONSORED RESEARCH

Not applicable

### PARTIES TO A JOINT RESEARCH AGREEMENT

Not Applicable

### REFERENCE TO APPENDIX

Not applicable

### FIELD OF THE INVENTION

The field of the present invention generally relates to data recorders, and more particularly, to data recorders for use with analyzing bodies of water in watersheds.

### BACKGROUND OF THE INVENTION

Fresh water lakes, ponds, rivers, and streams etc. are an important resource to culture and economy of their surrounding area, providing drinking water, irrigation to arable lands, recreational activities, and tourism. They can also home to a wide range of wildlife, including fish, mammals, and migratory and resident birds. Since the 1990s, fresh water bodies of water in the United States have been experiencing increased growth of cyanobacteria, commonly known as blue-green algae. This is a naturally occurring plant-like organism, found in shallow, warm, slow-moving waters that contain the toxin microystin. In freshwater environments, phosphorous and nitrogen are limiting nutrients, in that they either limit or excel an organism's growth. An excess of these nutrients in fresh water, known as nutrient loading, leads to an uncontrolled growth of cyanobacteria, often called an algal bloom.

Nutrient loading is in part caused by fertilizers (synthetic and organic) used in agricultural practices, urbanization and human waste, and household products containing phosphorous compounds, such as detergents. The excess dissolved phosphorous and nitrogen can enter nearby water systems by run off or leaching. This increases the growth of cyanobacteria and invasive aquatic species as they feed on dissolved nitrate and phosphate, producing murky green waters known as algal blooms. Algal blooms have caused problems across the United States, particularly in the Midwest and South, impacting human health and altering ecosystems. Human health is threatened if contaminated water is consumed as it can trigger gastrointestinal discomfort and even liver damage. In addition, algal blooms may prevent engagement in recreational activities such as boating, swimming and fishing, due to toxicity. This is economically damaging. Cyanobacteria blocks light for photosynthesis from reaching the bottom of the lake, affecting aquatic vegetation, and in turn, aquatic species that rely on the vegetation for food and nurseries. As algae decays, it consumes oxygen and releases carbon dioxide, causing mortalities of plants and animals. Dead zones are created in the water, which are areas of hypoxic conditions in which very few organisms can survive. In addition, invasive species, such as zebra and quagga mussels, that have entered the ecosystem by human activities, are perpetuating the issue by consuming edible algae, allowing the toxic algae to survive with less competition. Furthermore, heavy rains and warmer temperatures due to climate change are believed to be altering ecosystems and increasing runoff. The resulting changes in salinity and carbon dioxide levels further promote growth of algae.

To solve this problem, site-specific analysis, alterations, and management are necessary. Accordingly, there is a need for economical systems and methods for collecting data from watersheds.

### SUMMARY OF THE INVENTION

The present invention provides systems, devices, and methods which address at least one of the above-noted problems of the prior art. Disclosed herein is a watershed data recorder for obtaining data from a body of water comprising, in combination, a watercraft having at least one hull for moving along the body of water, a sensor pod includes a water contact sensor adapted to determine if the sensor is in the body of water, a pressure sensor adapted to determine the depth of the sensor pod, and a plurality of sensors for measuring water conditions, a motorized winch for selectively raising and lowering the sensor pod into and out of the body of water, and vertically through a full depth of the body of water, and a controller operatively connected to the winch to control movement of the sensor pod and operatively connected to a Global Positioning System (GPS) receiver to log positions of sensor pod movements.

Also disclosed herein is a watershed data recorder for obtaining data from a body of water that comprises, in combination, a watercraft having at least one hull for moving along the body of water, at least one fan propeller, or other means of propulsion, for selectively generating thrust for the watercraft, a sensor pod including a plurality of sensors for measuring water conditions, and a motorized winch carried by the watercraft for selectively raising and lowering the sensor pod into and out of the body of water, and vertically through a full depth of the body of water.

Further disclosed herein is a method for making a three-dimensional water quality attribute data collection for a body of water. The method comprises the steps of, in combination, propelling a watershed data recorder along a surface of the body of water, lowering a sensor pod into the water at a plurality of sensing locations along the body of water and logging a GPS location for each of the plurality of sensing locations, and obtaining water conditions using a plurality of sensors of the sensor pod at a plurality of depths at each of the plurality of sensing locations and logging water conditions and depth at each of the plurality of depths. Wherein the watershed data recorder comprises a watercraft having at least one hull for moving along the body of water, the sensor pod including a water contact sensor adapted to determine if the sensor pod is in the body of water, a pressure sensor adapted to determine the depth of the sensor pod, and the plurality of sensors for measuring water conditions, a motorized winch for selectively raising and lowering the sensor pod into and out of the body of water, and vertically through a full depth of the body of water, and a controller operatively connected to the winch to control movement of the sensor pod and operatively connected to a Global Positioning System (GPS) receiver to log positions of sensor pod movements;

From the foregoing disclosure and the following more detailed description of various preferred embodiments it will be apparent to those skilled in the art that the present invention provides a significant advance in the technology and art of data recorders for watersheds. Particularly, significant in this regard is the potential the invention affords for providing an economical, reliable and effective data recorder for a body of water that is durable, provides three-dimensional data, and provides repeatable data collection of the same site over time. Additional features and advantages of the invention will be better understood in view of the detailed description provided below.

### BRIEF DESCRIPTION OF THE DRAWINGS

These and further objects of the invention will become apparent from the following detailed description.

FIG. 1 is a perspective view of a watershed autonomous data collector according to the present invention.

FIG. 2 is a front elevational view of the watershed autonomous data collector of FIG. 1.

FIG. 3 is a port or left-side elevational view of the watershed autonomous data collector of FIGS. 1 and 2.

FIG. 4 is a stern or rear elevational view of the watershed autonomous data collector of FIGS. 1 to 3.

FIG. 5 is a starboard or right elevational view of the watershed autonomous data collector of FIGS. 1 to 4.

FIG. 6 is a top plan view of the watershed autonomous data collector of FIGS. 1 to 5.

FIG. 7 is a bottom plan view of the watershed autonomous data collector of FIGS. 1 to 6.

FIG. 8 is a side view of a sensor pod the watershed autonomous data collector of FIGS. 1 to 6.

FIG. 9 is a side view of a sensor module of the sensor pod FIG. 8.

FIG. 10 is a block diagram of electric/electronic systems of the watershed autonomous data collector of FIGS. 1 to 9.

FIG. 11 is top view of a body of water illustrating exemplary data collection points.

FIG. 12 is cross-sectional view of the body of water taken along line 12-12 of FIG. 11.

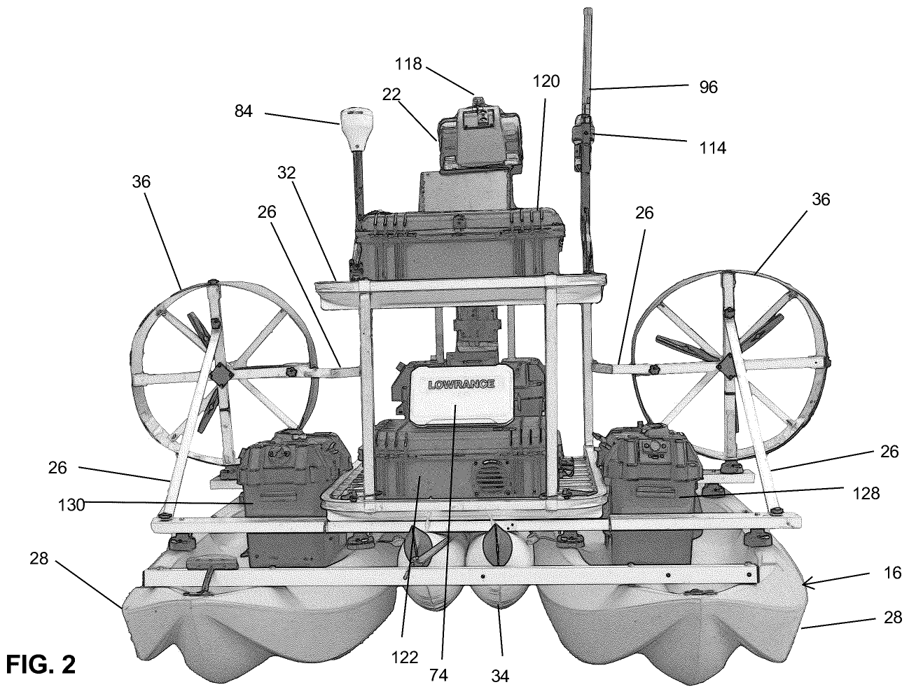

It should be understood that the appended drawings are not necessarily to scale, presenting a somewhat simplified representation of various preferred features illustrative of the basic principles of the invention. The specific design features of the watershed autonomous data collector as disclosed herein, including, for example, specific dimensions, orientations, locations, and shapes will be determined in part by the particular intended application and use environment. Certain features of the illustrated embodiments have been enlarged or distorted relative to others to facilitate visualization and clear understanding. In particular, thin features may be thickened, for example, for clarity or illustration. All references to direction and position, unless otherwise indicated, refer to the orientation of the watershed autonomous data collector illustrated in the drawings. In general, up or upward generally refers to an upward direction within the plane of the paper in FIG. 2 and down or downward generally refers to a downward direction within the plane of the paper in FIG. 2. Also in general, fore or forward generally refers to an outward direction out the plane of the paper in FIG. 2 and aft or rearward generally refers to an inward direction into the plane of the paper in FIG. 2.

### DETAILED DESCRIPTION OF CERTAIN PREFERRED EMBODIMENTS

It will be apparent to those skilled in the art, that is, to those who have knowledge or experience in this area of technology, that many uses and design variations are possible for the systems, devices, and methods disclosed herein. The following detailed discussion of various alternative and preferred embodiments will illustrate the general principles of the invention with regard to the specific application of watershed autonomous data collector configured for data collection of lakes, ponds, etc. Other embodiments suitable for other applications will be apparent to those skilled in the art given the benefit of this disclosure.

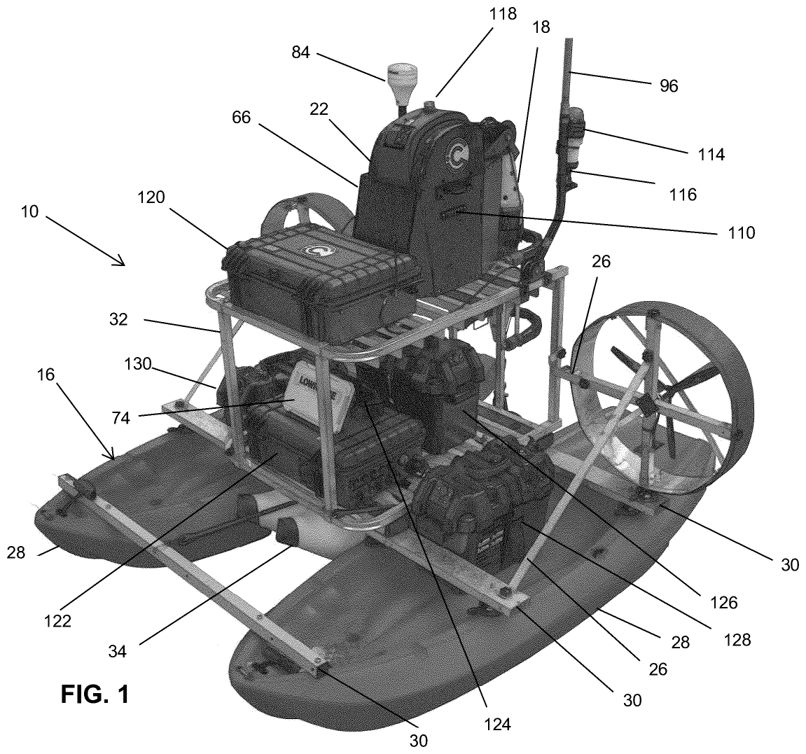

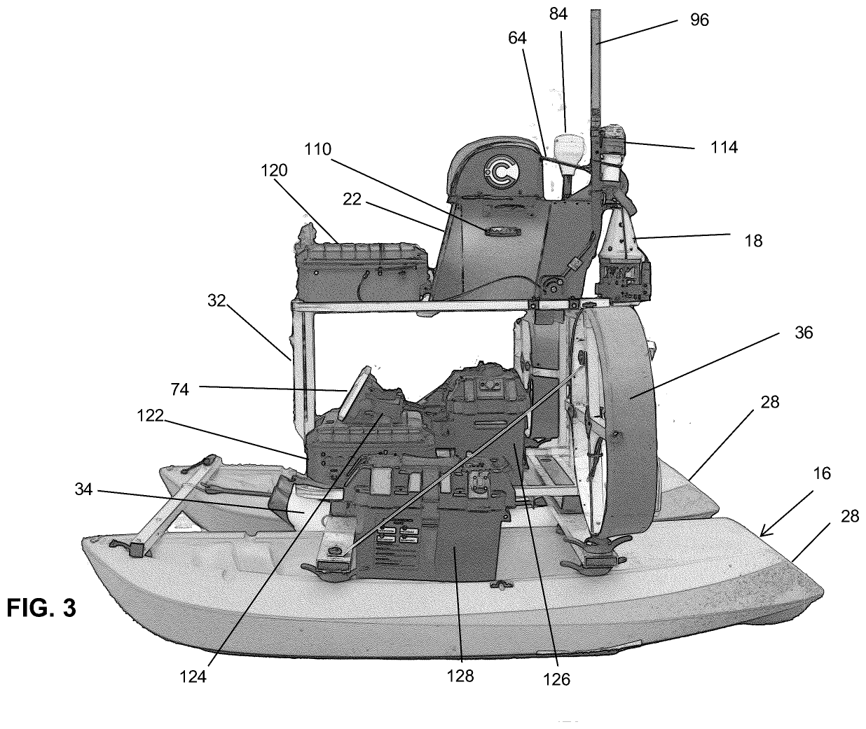

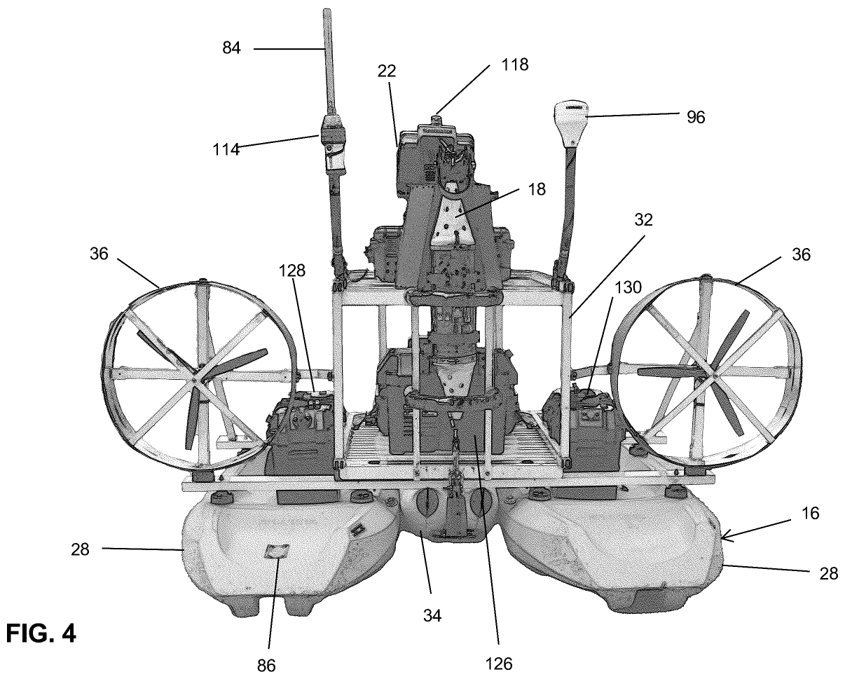

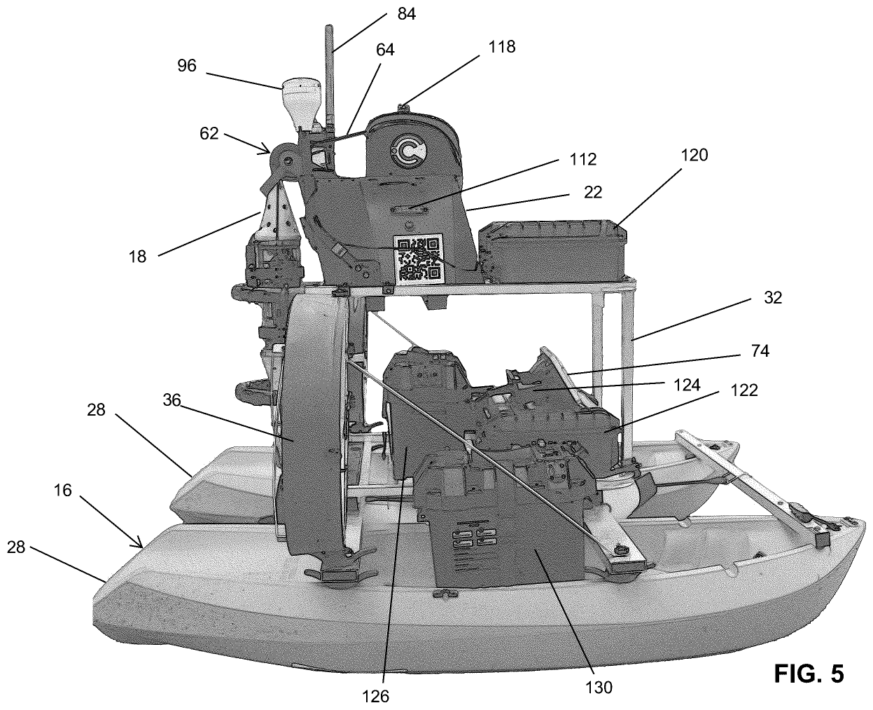

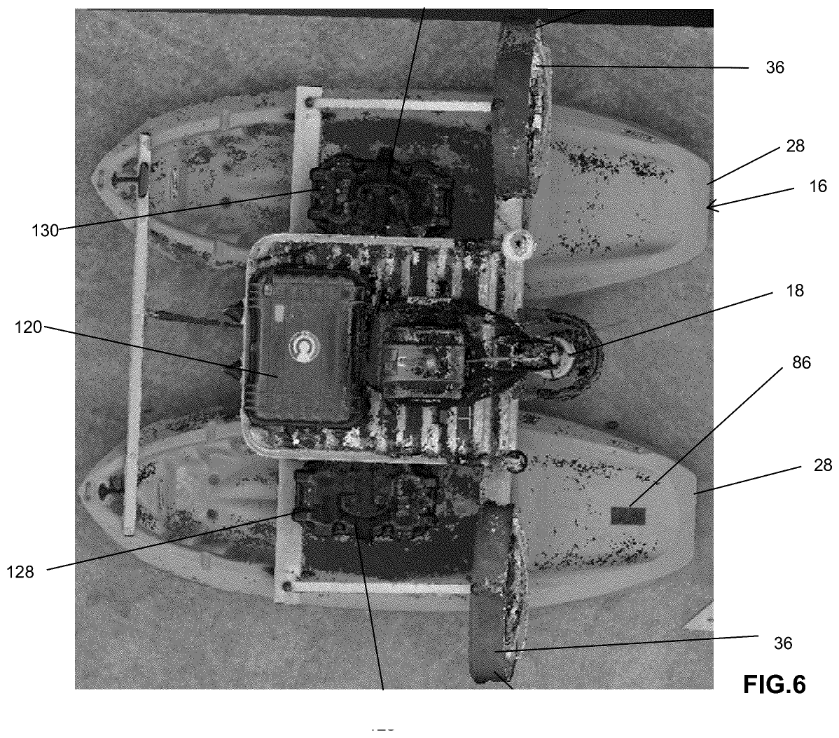

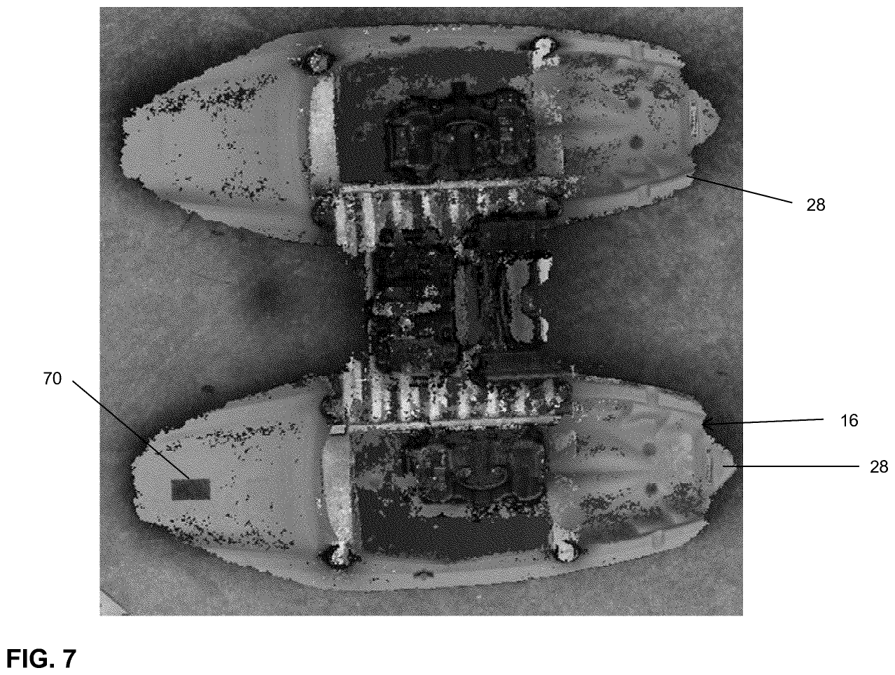

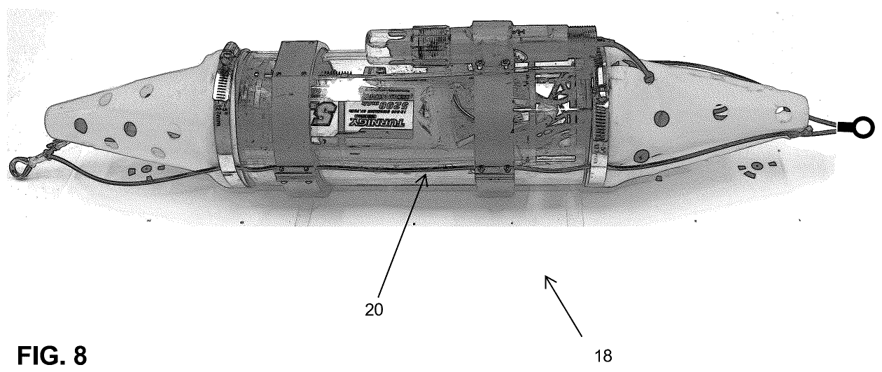

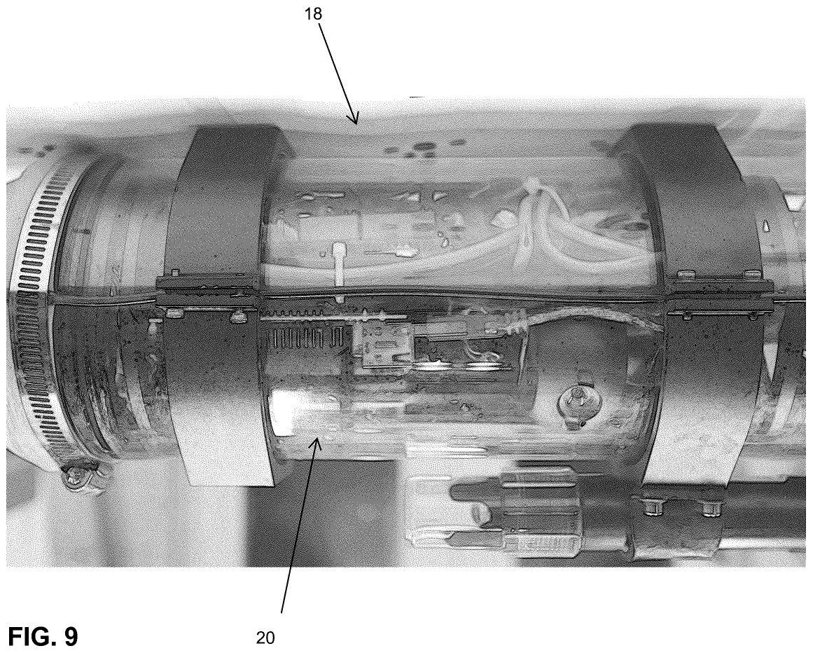

FIGS. 1 to 10 illustrate a watershed autonomous data recorder 10 for obtaining data from a body of water according to the present invention. The illustrated watershed autonomous data recorder 10 is configured for mapping subsurface topography and obtaining water quality data from the body of water which enables identification of the areas of highest concentration of waterborne pollutants/nutrients which correlate with the most likely sources of waterborne pollutants/nutrients into the body of water. The illustrated watershed autonomous data collector 10 obtains a three-dimensional environmental, physical and chemical water quality attribute data collection which creates a digital twin of the body water. This digital twin of the body of water establishes a common data environment for use in water modeling and analysis.

The illustrated watershed autonomous data recorder 10 has fully autonomous navigation with built-in auto collision avoidance with shorelines, shoreline infrastructure, heavy aquatic plant growth, other watercraft, and/or other obstacles it may encounter while collecting data with the aid of a Global Positioning System (GPS) and internal mapping. Onboard controllers provide navigation control, sensor deployment, data management and real-time communications via, for example but not limited to, satellite, LoRa, cellular, Bluetooth, Wi-Fi, RF telemetry, or the like. Alternatively, the illustrated watershed autonomous data recorder 10 be manually navigated similar to a radio-controlled watercraft and/or aircraft. It is noted that the watershed autonomous data recorder 10 can alternatively be provided with and controlled by any other suitable type of navigation,

The illustrated watershed autonomous data recorder 10 can be utilized to collect data in both non-tethered and tethered applications. In non-tethered operation, the watershed autonomous data collector 10 moves about the body of water for data collection over the entire body of water or any other desired portion of the body of water. The watershed autonomous data recorder 10 is moved about over a desired path on the body of water by a tangle-proof or non-tangling propulsion system 12 powered by a power system 14 having one or more onboard battery, solar cell, fuel cell, combustion engine, or other suitable power source. In tethered operation, the watershed autonomous data collector 10 is tethered or tied to a fixed location for data collection when near-shore, long term data collection is desired.

The illustrated watershed autonomous robotic data recorder 10 includes a watercraft 16 for moving along the body of water, the tangle-proof or non-tangling propulsion system 12 for selectively generating thrust to propel the watercraft 16 over the water, a deployable sensor pod 18 including a plurality of water condition sensors 20 for measuring water conditions, a motorized winch 22 for selectively lowering and raising the sensor pod 18 into and out of the body of water, and a controller 24 operatively connected to the motorized winch 22 to control movement of the sensor pod 18 and operatively connected to a Global Positioning System (GPS) receiver 88 to log positions of the sensor pod 18 movements.

The watercraft 16 includes at least one hull 28. The illustrated watercraft 16 is in the form of a catamaran having a pair of laterally spaced-apart hulls 28 secured together. The illustrated hulls 28 are kayaks such as, for example but not limited to, six-foot long plastic kayaks. The hulls 28 can alternatively have any other suitable form for navigation of a variety of types of water from littoral lagoons to larger less protected semi open water. Laterally extending between the hulls 28 are a plurality of beams or spars 30 spaced-apart in the forward-rearward direction and secured to each of the hulls 28. The illustrated watercraft 16 includes three of the beams or spars 30 but any other suitable quantity can alternatively be utilized. The beams or spars 30 are preferably secured with quick-attach mounts such as, for example but not limited to, Railblaza Cleatport quick attach mounts but can alternatively be secured in any other suitable manner. Also extending between the hulls 28 is a superstructure support frame 32 having lower and upper shelves for supporting equipment as described in more detail herein below. The illustrated superstructure support frame 32 is secured to two of the beams or spars 30 using Cleatport studs but can alternatively be secured to the spars 30 or directly to the hulls 28 in any other suitable manner. In the illustrated embodiment, supplemental flotation 34 is provided between the hulls 28 and below the superstructure support frame 32. The supplemental flotation 34 can be removed if not desired and/or needed. The illustrated watercraft 16 has an overall size of a length of about 6 feet, a width of about 5 feet and a height of about 4 feet. It is noted that the watercraft 16 can alternatively have any other suitable size and/or form such as, for example but not limited to, a single hull 28 or more than two hulls 28. It is also noted that the illustrated modular hull design provides the ability to customize the cross-section of the watercraft 16 in order to accommodate navigation on different sizes of bodies of water. For example, but not limited to, adding an additional hull 28 for larger bodies of water or eliminating a hull 28 for smaller bodies of water.

The tangle-proof or non-tangling propulsion system 12 can be in the form of at least one rearward-facing airboat fan or propulsion fan 36 that blows air in the rearward direction to forwardly propel the watercraft 16 along the water. The illustrated watercraft 16 includes a pair of the rearward-facing air boat fans or propulsion fans 36 which are laterally spaced-apart. Each propulsion fan 36 is secured to a separate one of the two hulls 28. The illustrated propulsion fans 36 are also provide with a pair of supports struts 26 for stability. Each illustrated propulsion fan 36 includes a propeller rotated by an electric motor powered by an electric battery. The propulsion fans 36 can alternatively be rotated in any other suitable manner. The tangle-proof propulsion system 12 is advantageous where there is a large amount of vegetation, algae, etc. in the water which can interfere with a typical boat propeller by tangling or wrapping around the boat propeller. It is noted that the non-tangling propulsion system 12 can alternatively be of any other suitable type such as, for example but not limited to, at least one water jet or the like. Alternatively, any other suitable type of propulsion system can be utilized.

The illustrated deployable sensor pod or dipping sensor 18 includes a water contact sensor 38 adapted to determine if the sensor pod 18 is in the body of water, a pressure sensor or transducer 40 adapted to determine the depth of the sensor pod 18 in the water, and the plurality of water condition sensors 20 for measuring various water conditions. The illustrated plurality of water condition sensors 20 includes a temperature sensor 42, a turbidity sensor 44, a dissolved O2 sensor 46, a pH sensor 48, a TDS sensor 50, and a pair of accelerometer/gyro sensors 52. The plurality of water condition sensors 20 can alternatively be of any other suitable type, such as nitrite and phosphate sensors, and/or quantity depending on the particular body of water and the desired water condition data desired. The illustrated sensor pod 18 also includes a video camera 54 for providing underwater video of the sensors 20 and/or other underwater visual conditions. The illustrated sensor pod 18 further includes at least one battery 56 so that the sensor pod 18 is self-powered and does not need any power cords or the like extending to it from the watercraft 16. The illustrated sensor pod 18 additionally includes at least one single-board microcontroller 58 for connecting the sensors to another at least one single-board microcontroller 60 controlling operation of the sensor pod components and storing data. The illustrated single-board microcontrollers 58, 60 are an Arduino Uno microcontroller and a Rasberry Pi single-board microprocessor respectively, but any other suitable microcontrollers can alternatively be utilized. It is noted that the sensor pod 18 can alternatively have any other suitable configuration.

A sensor pod support system 62 includes the motorized winch 22 driven by an electric motor to selectively raise and lower the sensor pod 18. The electric motor is driven by an electric power supply such as, for example but not limited to, a battery or the like. The electric motor turns a reel to wind and unwind a flexible line 64 such as, for example but not limited to, a nylon rope, cord or the like thereon with the sensor pod 18 secured to the free end of the flexible line 64. The illustrated electric winch 22 is supported by a winch tower or mount 66 secured at a rear of the top shelf of the superstructure support frame 32 so that the sensor pod 18 is raised and lowered between the rear ends of the hulls 28. The illustrated winch tower 66 is secured to the superstructure support frame 32 using hitch pins through the metal support frame 32 but it is noted that winch tower 66 can alternatively be secured in any other suitable manner. It is noted that the sensor pod support system 62 can alternatively have any other suitable configuration

The illustrated watershed autonomous data collector 10 also includes a sonar system 68 adapted to produce three-dimensional scans of underwater terrain. The illustrated sonar system 68 includes a sonar depth transducer 70 located on an underside of the port-side hull 28, at a forward end of the port side hull 28. The sonar depth transducer 70 is used to measure the underwater topography of the bottom of the water. The transducer 70 transmits sound waves into the water, receives the reflections (echoes) back, and interprets what is below the surface of the water. A chart plotter 74 and GPS receiver 76 are also included to record and identify locations of the scans. It is noted that the sonar system 68 can alternatively have any other suitable configuration. It is also noted that the watershed autonomous data collector 10 also includes a sonar ping or side scan transducer 86 as described in more detail below

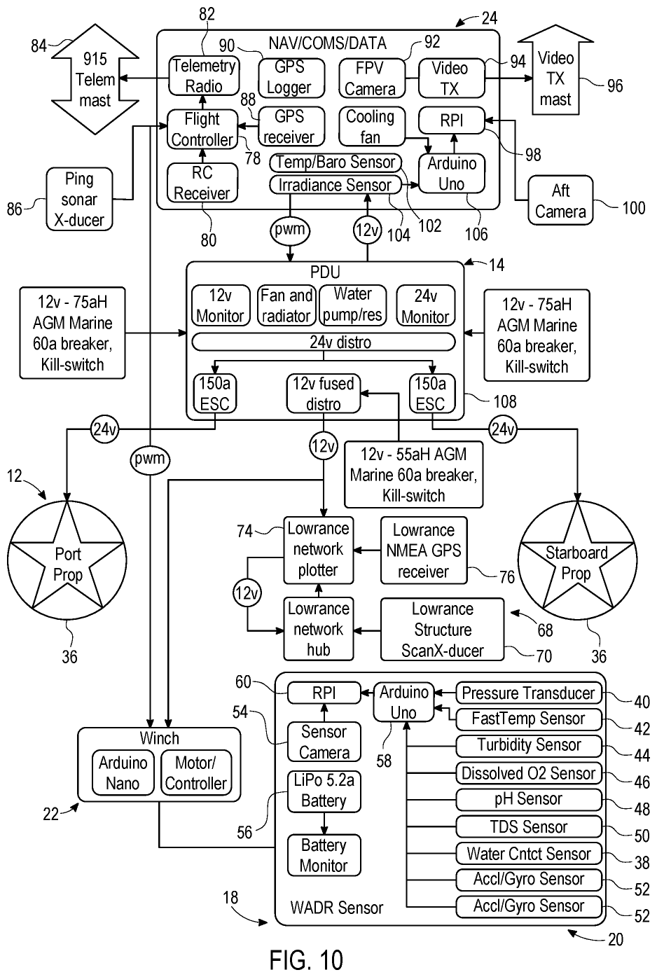

As best shown in FIG. 10, the illustrated Navigation/Communication/Data controller 24 includes a flight controller 78 in communication with a RC receiver 80 and a telemetry radio 82 to provide remote and/or autonomous control of the watershed autonomous data recorder 10. The telemetry radio 82 is provided with a 915 MHz telemetry mast 84. Alternatively, telemetry can be provided by utilizing satellite, cellular, lora, Bluetooth or wi-fi data transceivers. A sonar ping or side scan transducer 86 is in communication with the flight controller 78. The illustrated ping or side scan transducer 86 is located at a rear end of the port-side hull 28, on an underside of the port-side hull 28. The ping transducer 86 perform measurements between moving or stationary objects by triggering ultrasonic bursts (well above human hearing) and then “listening” for an echo return pulse. The transducer measures the time required for the echo return, and returns this value to the flight controller 78. This distance information is used by the flight controller 78 for robotic navigation and crash avoidance. GPS receiver 88 with GPS logger 90 is also in communication with the flight controller 78. The controller 24 also includes a first-person view (FPV) camera 92 provided with a video transmitter 94 and video transmitter mast 96. A single-card microprocessor 98 (Rasberry Pi) is provided to receive the input from an aft video camera 100 and also a temperature/barometer sensor 102 and a Irradiance sensor 104 via another single-card microcontroller 106 (Arduino Uno). The controller 24 receives power from a power distribution unit 108. The controller is also in communication with the motorized winch 22 for controlling operation of the motorized winch 22. It is noted that the controller 24 can alternatively have any other suitable configuration.

As best shown in FIG. 10, the illustrated power system 14 includes the power distribution unit 108 configured to provide 5 volt to 48 volt electric power to the various components as described herein. It is noted that the power system 14 can alternatively have any other suitable configuration.

The illustrated watershed autonomous data recorder 10 includes a standard red port-side navigation light 110, a standard green starboard-side navigation light 112, and a standard white stern navigation light 114. The illustrated port and starboard side navigation lights 110, 112 are located at the port and starboard sides of the winch tower 66 respectively. The illustrated stern navigation light 114 is located at the top of a mast 116 secured to the back of the superstructure support frame 32. It is noted that the navigation lights 110, 112, 114 can alternatively have any other suitable configuration. Additionally an anti-collision strobe light 118 is provided at the atop of the winch tower 66. It is noted that the anti-collision strobe 118 can alternatively have any other suitable configuration.

The illustrated watershed autonomous data recorder 10 includes a plurality of water-proof storage boxes or containers for storing various components and equipment in a water free environment. The illustrated storage boxes are retained in place to the watercraft 16 using EPDM bungie straps or the like but can alternatively be retained in any other suitable manner. The illustrated storage boxes include: a navionic/communication/topside sensor logging box 120 located at the front of the top shelf of the super structure support frame 32; a power distribution box 122 located at the front of the bottom shelf of the superstructure support frame 32; a structure scan 3D module box 124 located on top of the power distribution box 122; a navionics power supply box 126 located at the rear of the bottom shelf of the superstructure support frame 32; a port-side propulsion power supply box 128 located on the port-side hull 28 in front of the port-side propulsion fan 36; and a starboard-side propulsion power supply box 130 located on the starboard-side hull 28 in front of the starboard-side propulsion fan 36. It is noted that there can alternatively be any other suitable quantity, size and/or configuration of the storage boxes or the components can be stored in any other suitable manner.

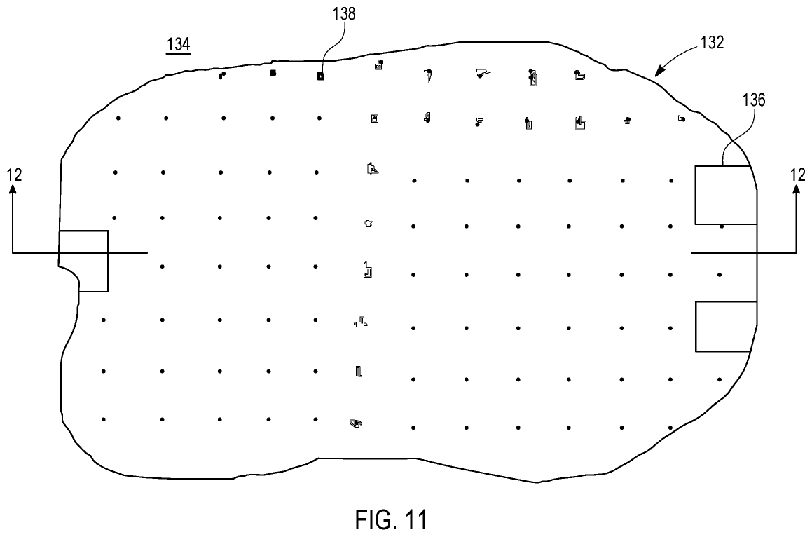

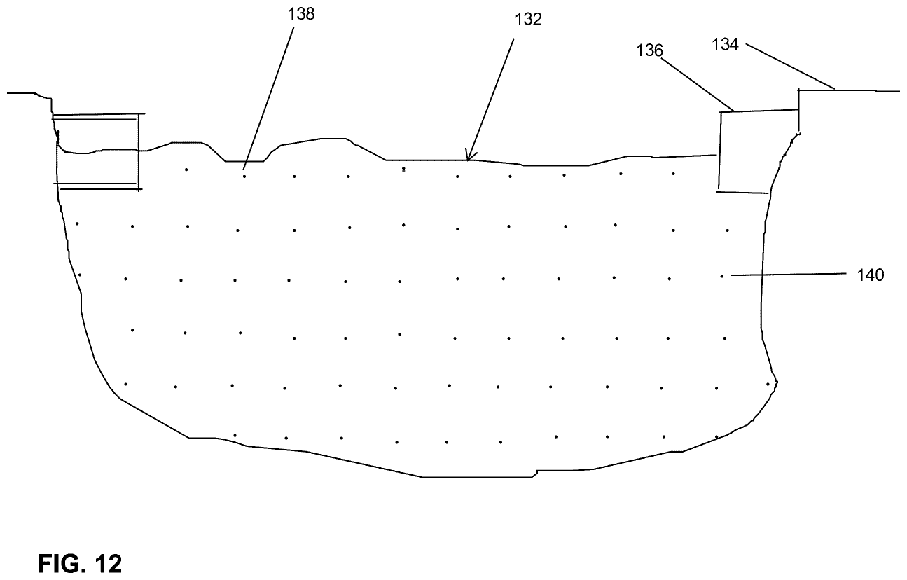

FIGS. 11 and 12 illustrate an exemplary body of water 132 having a shoreline 134 with plurality of water inlets/outlets 136. The above-described watershed autonomous data collector 10 can be utilized to collect water quality attribute data for the body of water 132. A method for making a three-dimensional water quality attribute data collection for the body of water 132 includes the steps of autonomously propelling the watershed autonomous data recorder 10 along a surface of the body of water 132 over a predetermined path to cover the entire body of water 132 or substantially all of the body of water 132. At predetermined plurality sensing locations 138 along the path, the watershed autonomous data recorder 10 is stopped and the sensor pod 18 is lowered by the motorized winch 22. While the water sensor 38 indicates the sensor pod 18 is out of the water, sensor readings are taken once a minute. Once the water contact sensor 38 indicates that the sensor pod 18 is in the water, sensor readings are taken once every second for the full water column until the sensor pod 18 stops. The sensor pod 18 is then raised at a speed of one foot per second while taking sensor readings every second. Thus, water conditions using the plurality of water condition sensors 20 is obtained at a plurality of depths 140 about one foot apart at each of the plurality of sensing locations 138. It is noted that any other suitable rate of measuring water conditions can alternatively be used. The water conditions (as determined by the plurality of water condition sensors 20) and the depth (as determined by the pressure sensor 40) at each of the plurality of depths 140 is logged. A GPS location for each of the plurality of sensing locations 138 is also determined and logged. Thus, the process can be accurately repeated at a later date to determine changes over time. With this method, three-dimensional environmental, physical, and chemical water quality attribute data is collected which creates a digital twin of the body of water 132 and establishes a common data environment for use in water modeling and analysis. This method also provides the ability to autonomously measure waterborne pollutants/nutrients, utilize search algorithms to seek out areas of their highest concentrations, and identify most the likely location(s) of their entry into the body of water 132.

It is noted that each of the features of the various disclosed embodiments of the present invention can be utilized in any combination with each of the other disclosed embodiments of the present invention.

From the above disclosure it can be appreciated that the watershed autonomous robotic data recorders 10 according to the present invention provide a first of its kind bathymetric scalable drone capable of fully autonomous mapping of lake/pond infrastructure and water quality sampling in a true three-dimensional data collection process. Additionally, the watershed autonomous robotic data recorders 10 according to the present invention provide: autonomous programmable operation; programmable operation which enable timeline trending of repeated water quality measurements from the exact same spots in the bodies of water; three-dimensional water quality measurements from a mobile platform; a high density of water body quality measurements—hundreds of thousands of data points collected to measure non-homogenous characteristics of water bodies; customizable water quality sensor loads; tangle-proof propulsion which enables navigation even through dense lake/pond vegetation; a full view of the micro-watershed that completely incorporates the body of water when combining aerial and subsurface water topography; and measurement of hard and soft lake/pond bottoms and calculations of lake/pond sediment to water ratios.

The preferred embodiments of this invention can be achieved by many techniques and methods known to persons who are skilled in this field. To those skilled and knowledgeable in the arts to which the present invention pertains, many widely differing embodiments will be suggested by the foregoing without departing from the intent and scope of the present invention. The descriptions and disclosures herein are intended solely for purposes of illustration and should not be construed as limiting the scope of the present invention.

What is claimed is:

1. A watershed data recorder for obtaining data from a body of water comprising, in combination:
a watercraft having at least one hull for moving along the body of water;
a sensor pod includes a water contact sensor adapted to determine if the sensor is in the body of water, a pressure sensor adapted to determine the depth of the sensor pod, and a plurality of sensors for measuring water conditions;
a motorized winch for selectively raising and lowering the sensor pod into and out of the body of water, and vertically through a full depth of the body of water; and
a controller operatively connected to the winch to control movement of the sensor pod and operatively connected to a Global Positioning System (GPS) receiver to log positions of sensor pod movements.

2. The watershed data recorder according to claim 1, wherein the watercraft is configured for autonomous navigation and collision avoidance using GPS, and internal mapping and environmental sensors.

3. The watershed data recorder according to claim 1, wherein the plurality of sensors for measuring water conditions includes at least one of a temperature sensor, a turbidity sensor, a dissolved O2 sensor, a pH sensor, a TDS sensor, and accelerometer/gyro sensors.

4. The watershed data recorder according to claim 1, wherein the sensor pod includes a camera.

5. The watershed data recorder according to claim 1, wherein the sensor pod is self-powered by a battery.

6. The watershed data recorder according to claim 1, further a three-dimensional sonar transducer adapted to produce three-dimensional scans of underwater terrain.

7. The watershed data recorder according to claim 1, further comprising a tangle-proof propulsion system for selectively generating thrust for the watercraft.

8. The watershed data recorder according to claim 1, wherein the watercraft includes at least two laterally spaced apart hulls secured together.

9. The watershed data recorder according to claim 1, wherein the watercraft has at least two hulls and is modular so that a cross-section of the watercraft can be customized by adding and removing at least one of the at least two hills to accommodate navigation on different sizes the body of water.

10. A watershed data recorder for obtaining data from a body of water comprising, in combination:
a watercraft having at least one hull for moving along the body of water;
at least one fan propeller for selectively generating thrust for the watercraft;
a sensor pod including a plurality of sensors for measuring water conditions; and
a motorized winch carried by the watercraft for selectively raising and lowering the sensor pod into and out of the body of water, and vertically through a full depth of the body of water.

11. The watershed data recorder according to claim 10, wherein the watercraft includes a pair of laterally spaced-apart fan propellers for selectively generating thrust for the watercraft.

12. The watershed data recorder according to claim 10, wherein the watercraft is configured for autonomous navigation and collision avoidance using GPS, and internal mapping and environmental sensors.

13. The watershed data recorder according to claim 10, wherein the sensor pod further includes a water contact sensor adapted to determine if the sensor is in the body of water, and a pressure sensor adapted to determine the depth of the sensor pod.

14. The watershed data recorder according to claim 10, further comprising a controller operatively connected to the winch to control movement of the sensor pod and operatively connected to a Global Positioning System (GPS) receiver to log positions of sensor pod movements.

15. The watershed data recorder according to claim 10, wherein the plurality of sensors for measuring water conditions includes at least one of a temperature sensor, a turbidity sensor, a dissolved O2 sensor, a pH sensor, a TDS sensor, and accelerometer/gyro sensors.

16. The watershed data recorder according to claim 10, wherein the sensor pod includes a camera.

17. The watershed data recorder according to claim 10, wherein the sensor pod is self-powered by a battery.

18. The watershed data recorder according to claim 1, further a three-dimensional sonar transducer adapted to produce three-dimensional scans of underwater terrain.

19. The watershed data recorder according to claim 1, wherein the watercraft has at least two hulls and is modular so that a cross-section of the watercraft can be customized by adding and removing at least one of the at least two hulls to accommodate navigation on different sizes the body of water.

20. A method of making a three-dimensional water quality attribute data collection for a body of water, the method comprising the steps of, in combination:
propelling a watershed data recorder along a surface of the body of water, wherein the watershed data recorder comprises a watercraft having at least one hull for moving along the body of water, a sensor pod including a water contact sensor adapted to determine if the sensor pod is in the body of water, a pressure sensor adapted to determine the depth of the sensor pod, and a plurality of sensors for measuring water conditions, a motorized winch for selectively raising and lowering the sensor pod into and out of the body of water, and vertically through a full depth of the body of water, and a controller operatively connected to the winch to control movement of the sensor pod and operatively connected to a Global Positioning System (GPS) receiver to log positions of sensor pod movements;
lowering the sensor pod into the water at a plurality of sensing locations along the lake and logging a GPS location for each of the plurality of sensing locations; and
obtaining water conditions using the plurality of sensors at a plurality of depths at each of the plurality of sensing locations and logging water conditions and depth at each of the plurality of depths.
